(19) United States
(12) Patent Application Publication
(10) Pub. No.: US 2026/0115447 A1
(43) Pub. Date: Apr. 30, 2026

(54) PIVOTALLY-DRIVEN FLEXIBLE MEMBRANE BLOOD PUMP

(71) Applicants: NANJING DRUM TOWER HOSPITAL, Nanjing (CN); NANJING TECH UNIVERSITY, Nanjing (CN)

(72) Inventors: Can XU, Nanjing (CN); Dongjin WANG, Nanjing (CN)

(21) Appl. No.: 19/163,301

(22) PCT Filed: Oct. 25, 2024
(86) PCT No.: PCT/CN2024/127193

§ 371 Date: Sep. 8, 2025

(30) Foreign Application Priority Data
Mar. 25, 2024 (CN) 202410338623.3

(51) Int. Cl. A61M 60/268; A61M 60/178; A61M 60/859

(52) U.S. Cl. CPC A61M 60/268; A61M 60/178; A61M 60/859

(57) ABSTRACT

The present invention discloses a pivotally-driven flexible membrane blood pump, and the technical solutions are as follows: The pivotally-driven flexible membrane blood pump includes a pump body, and an inlet connection and an outlet connection that communicate with the pump body. Ends of the inlet connection and the outlet connection communicate with flexible pipes via fastening mechanisms. The fastening mechanism includes a clamping groove formed in the inlet connection. Multiple pressing slots that communicate with the clamping groove are formed in a surface of the inlet connection. A first limiting ring and a second limiting ring are fixedly connected to an outer side of the inlet connection respectively. An external thread is formed on the inlet connection between the first limiting ring and the second limiting ring. Technical effects of the present invention are as follows: by cooperation between the movable pipe and the external thread, the problem that both inlet and outlet connections of existing artificial blood pumps have no fastening mechanism for fastening flexible pipes, and the flexible pipe can only be clamped into the two connections by relying on the flexibility of the flexible pipe when connected, resulting in poor stability between the flexible pipe and the connections is solved.

### TECHNICAL FIELD

The present invention relates to the technical field of medical equipment, and in particular to a pivotally-driven flexible membrane blood pump.

### BACKGROUND

Currently, heart failure patients increase year by year in China. One of effective treatments for advanced heart failure is the surgical implantation of mechanical ventricular assist devices, primarily left ventricular assist devices. However, most left ventricular assist devices have a relatively large size, and need to perform highly invasive and high-risk surgery, and therefore are usually limited to be used only in end-stage heart failure patients. For patients not reaching the end-stage heart failure, a relatively small-sized pump may be implanted to avoid these risks and reduce perioperative morbidity. However, standard blood pumps that can be used in the clinic are mainly divided into axial-flow pumps and centrifugal pumps, both of which belong to rotary-type pumps. High-speed rotation of rotors of these bumps may cause damage to blood components, leading to hemolysis, thrombosis and bleeding complications. In addition, the blood pumps run at a fixed speed, which may reduce pulse pressure in patients during clinical use, and consequently, may cause complications related to continuous non-physiological blood pumping, such as gastrointestinal bleeding, aortic valve insufficiency, or stroke. Therefore, researchers develop a pulsation algorithm for such rotation-type pumps, to improve flushing, thereby reducing thrombosis and the like.

In the prior art, both inlet and outlet connections of existing artificial blood pumps have no fastening mechanism for fastening flexible pipes. The flexible pipe can only be clamped into the two connections by relying on the flexibility of the flexible pipe when connected, resulting in poor stability between the flexible pipe and the connections. Therefore, it is very necessary to apply a pivotally-driven flexible membrane blood pump to the existing field of artificial blood pumps.

### SUMMARY

An objective of the present invention is to provide a pivotally-driven flexible membrane blood pump, which has the advantages of: by cooperation between the movable pipe and the external thread, the problem that both inlet and outlet connections of existing artificial blood pumps have no fastening mechanism for fastening flexible pipes, and the flexible pipe can only be clamped into the two connections by relying on the flexibility of the flexible pipe when connected, resulting in poor stability between the flexible pipe and the connections is solved.

The above technical objective of the present invention is achieved through the following technical solutions: A pivotally-driven flexible membrane blood pump includes a pump body, and an inlet connection and an outlet connection that communicate with the pump body. Ends of the inlet connection and the outlet connection communicate with flexible pipes via fastening mechanisms.

The fastening mechanism includes a clamping groove formed in the inlet connection. Multiple pressing slots that communicate with the clamping groove are formed in a surface of the inlet connection. A first limiting ring and a second limiting ring are respectively fixedly connected to an outer side of the inlet connection. An external thread is formed on the inlet connection between the first limiting ring and the second limiting ring. An inner cavity is formed in the inlet connection. Multiple mounting pipes are arranged on an inner side of the inner cavity. An inserting rod is movably connected to one end of the mounting pipe. An operating plate is movably connected to one end of the inserting rod far from the mounting pipe.

The inlet connection is provided with a clamping mechanism.

The clamping mechanism includes an adjusting spring connected to the mounting pipe and the inserting rod. Two ends of the adjusting spring are connected to the operating plate and the inner cavity respectively. A movable pipe is mounted on the operating plate. One end of the movable pipe movably penetrates the clamping groove. Multiple connecting blocks are fixedly connected to the movable pipe. A fixing block is connected to an end of the connecting block close to the inlet connection. The fixing block is provided with a pressing mechanism.

The pivotally-driven flexible membrane blood pump is further configured such that: The pressing mechanism includes a placing groove formed in the fixing block. A guiding rod is slidingly connected in the placing groove. A clamping block is connected to one end of the guiding rod. An arc-shaped block is arranged at one end of the guiding rod far from the clamping block. Multiple telescoping springs are fixedly connected between the arc-shaped block and the fixing block.

The pivotally-driven flexible membrane blood pump is further configured such that: The inlet connection is further provided with an adjusting mechanism. The adjusting mechanism includes a threaded sleeve screwed to the external thread. The threaded sleeve is positioned between the first limiting ring and the second limiting ring. A connecting disk is connected to one end of the threaded sleeve. A connecting pipe is fixedly connected to the connecting disk. Multiple connecting rods are arranged on the connecting disk. A mounting rod is connected to one end of the connecting rod far from the connecting disk. A pushing pipe is connected to one end of the mounting rod far from the connecting rod.

The pivotally-driven flexible membrane blood pump is further configured such that: The connecting rod is positioned on an outer side of the second limiting ring. The mounting rod is positioned on one side of the second limiting ring.

The pivotally-driven flexible membrane blood pump is further configured such that: The inlet connection is further provided with a pulling mechanism. The pulling mechanism includes moving ports symmetrically formed in the connecting pipe. Assembly blocks are symmetrically and fixedly connected to an outer side of the connecting pipe. An inserting slot is formed in one end of the assembly block. A first clamping hole and a second clamping hole that communicate with the inserting slot are formed in a surface of the assembly block.

The pivotally-driven flexible membrane blood pump is further configured such that: The pulling mechanism includes an inserting block connected to the inserting slot. A guiding block is arranged at one end of the inserting block. The guiding block performs movable assembly corresponding to the moving port. An extension pipe is jointly connected between the two guiding blocks. The extension pipe is positioned between the pushing pipe and the connecting pipe.

The pivotally-driven flexible membrane blood pump is further configured such that: The pulling mechanism further includes an inner convex ring fixedly connected to an inner wall of the extension pipe. An operating cavity is formed in the inserting block. An elastic spring is fixedly connected to the operating cavity. A pushing plate is arranged at one end of the elastic spring. A telescoping button is mounted on the pushing plate. The telescoping button penetrates and is connected into the inserting block.

The present invention has the following beneficial effects:
1. According to the present invention, when the inlet connection and the outlet connection are clamped into a flexible pipe respectively, one end of the flexible pipe is positioned in the clamping groove. The threaded sleeve is rotated and moved on the external thread, to make the connecting rod drive the pushing pipe on the mounting rod to rotate and move, so that the pushing pipe can push the connecting block to move. The connecting block is pushed to move the movable pipe on the inner cavity. One end of the inserting rod moves inside of the mounting pipe, and the adjusting spring is extended. In this case, the connecting block and an inner wall of the clamping groove clamp a surface of the flexible pipe, thereby improving operation convenience.
2. According to the present invention, the fixing block on the connecting block is moved toward the pressing slot until the clamping block is positioned on one side of the pressing slot. The telescoping button on the second clamping hole is pressed, so that the elastic spring contracts. The inserting block is moved in the inserting slot, so that the telescoping button is changed to be clamped in the first clamping hole. In this case, the inner convex ring on the extension pipe applies a force on the arc-shaped block, so that the arc-shaped block drives the guiding rod to move on the fixing block. The telescoping spring contracts, and the guiding rod drives the clamping block to clamp the surface of the flexible pipe into the pressing slot, thereby increasing the stability between the flexible pipe and the inlet connection and the outlet connection respectively.

### BRIEF DESCRIPTION OF THE DRAWINGS

FIG. 1 is a schematic structural diagram of a pivotally-driven flexible membrane blood pump according to the present invention;

FIG. 2 is a schematic structural diagram of a fastening mechanism and a pipe assembly according to the present invention;

FIG. 3 is a cross-sectional schematic structural diagram of a fastening mechanism and a pipe assembly according to the present invention;

FIG. 4 is an exploded schematic structural diagram of an adjusting mechanism and a clamping mechanism according to the present invention;

FIG. 5 is a cross-sectional schematic structural diagram of a clamping mechanism according to the present invention;

FIG. 6 is a cross-sectional schematic structural diagram of an adjusting mechanism according to the present invention;

FIG. 7 is a cross-sectional schematic structural diagram of a pulling mechanism according to the present invention; and

FIG. 8 is a schematic structural diagram of a pressing mechanism according to the present invention.

Reference numerals: 10, pump body; 11, inlet connection; 12, outlet connection; 13, flexible pipe; 20, clamping groove; 21, pressing slot; 22, first limiting ring; 23, threaded sleeve; 30, connecting pipe; 31, moving port; 32, guiding block; 33, inserting block; 40, assembly block; 41, extension pipe; 42, inner convex ring; 43, connecting disk; 50, inner cavity; 51, mounting pipe; 52, inserting rod; 53, operating plate; 60, adjusting spring; 61, movable pipe; 62, connecting block; 63, fixing block; 70, second limiting ring; 71, connecting rod; 72, mounting rod; 73, pushing pipe; 80, external thread; 81, inserting slot; 82, first clamping hole; 83, second clamping hole; 90, operating cavity; 91, elastic spring; 92, pushing plate; 93, telescoping button; 94, placing groove; 95, guiding rod; 96, clamping block; 97, arc-shaped block; and 98, telescoping spring.

### DETAILED DESCRIPTION

The present invention will be further described in detail with reference to the accompanying drawings.

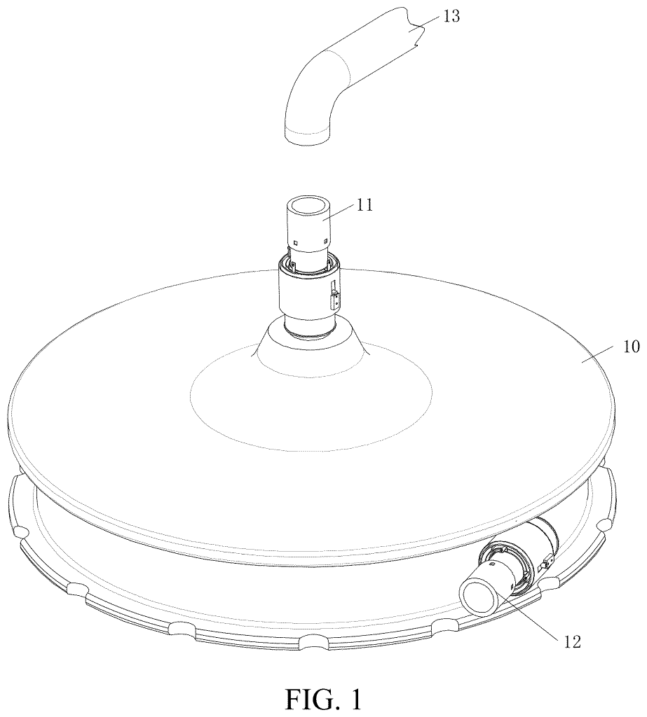

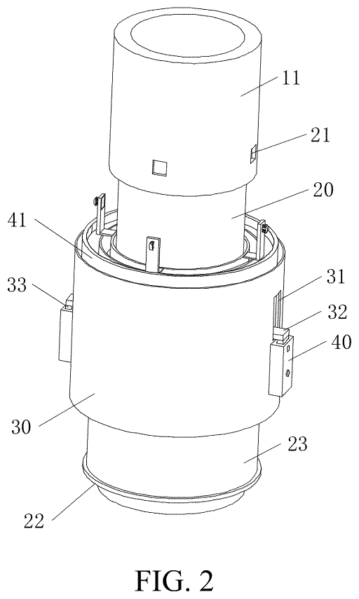

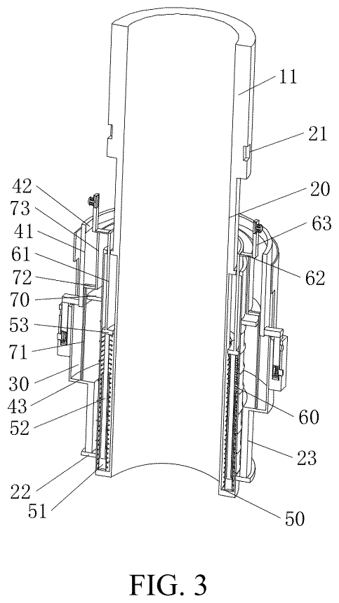

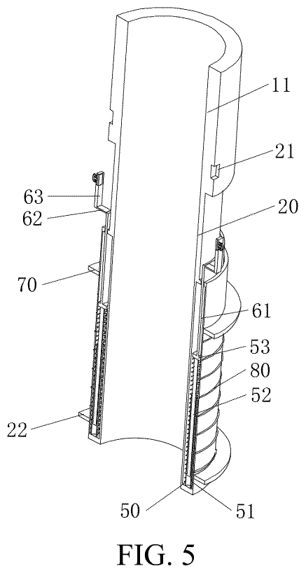

Embodiment: A pivotally-driven flexible membrane blood pump, referring to FIG. 1 to FIG. 5, includes a pump body 10 and a flexible pipe 13. The pump body 10 is fixedly assembled with an inlet connection 11. The pump body 10 is fixedly assembled with an outlet connection 12. Both the inlet connection 11 and the outlet connection 12 are clamped with flexible pipes 13. Both the inlet connection 11 and the outlet connection 12 are provided with clamping grooves 20. One end of the flexible pipe 13 is positioned in the clamping groove 20. Both the inlet connection 11 and the outlet connection 12 are provided with multiple pressing slots 21. The pressing slots 21 are positioned on one side of the clamping groove 20. Both the inlet connection 11 and the outlet connection 12 are fixedly assembled with first limiting rings 22. Both the inlet connection 11 and the outlet connection 12 are fixedly assembled with second limiting rings 70. The first limiting ring 22, the second limiting ring 70, and the pressing slots 21 are positioned on two sides of the clamping groove 20 respectively. An external thread 80 is fixedly assembled on the inlet connection 11 and the outlet connection 12, and the external thread 80 is positioned between the second limiting ring 70 and the first limiting ring 22. Both the inlet connection 11 and the outlet connection 12 are provided with inner cavities 50. The inner cavity 50 is fixedly assembled with multiple mounting pipes 51. One end of the mounting pipe 51 is movably assembled with an inserting rod 52. One end of the inserting rod 52 moves inside the mounting pipe 51. One end of the inserting rod 52 is fixedly assembled with an operating plate 53. An adjusting spring 60 is fixedly assembled between the operating plate 53 and the inner cavity 50. The mounting pipe 51 and the inserting rod 52 are assembled on an inner side of the adjusting spring 60. A movable pipe 61 is fixedly assembled on the operating plate 53. One end of the movable pipe 61 movably penetrates the clamping groove 20. Multiple connecting blocks 62 are fixedly assembled on the movable pipe 61. The connecting blocks 62 are positioned in the clamping groove 20. One end of the connecting block 62 is fixedly assembled with a fixing block 63. The fixing blocks 63 are positioned on one side of the inlet connection 11 and one side of the outlet connection 12 respectively. When the inlet connection 11 and the outlet connection 12 are clamped into a flexible pipe 13 respectively, one end of the flexible pipe 13 is positioned in the clamping groove 20. The connecting block 62 is pushed to move the movable pipe 61 on the inner cavity 50. One end of the inserting rod 52 moves inside of the mounting pipe 51, and the adjusting spring 60 is extended. In this case, the connecting block 62 and an inner wall of the clamping groove 20 clamp a surface of the flexible pipe 13, and the fixing block 63 on the connecting block 62 moves toward the pressing slot 21.

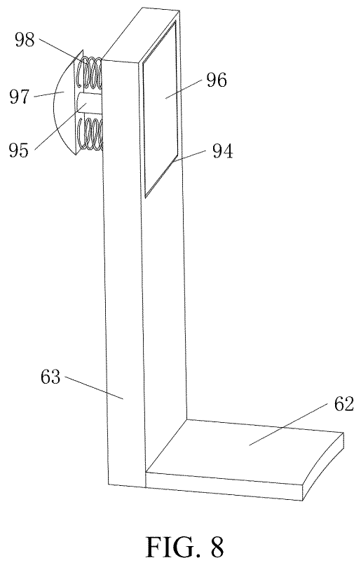

Referring to FIG. 8, a placing groove 94 is formed in the fixing block 63. A guiding rod 95 is movably assembled on the fixing block 63. One end of the guiding rod 95 movably penetrates the placing groove 94. One end of the guiding rod 95 is fixedly assembled with a clamping block 96 that moves on the placing groove 94. The other end of the guiding rod 95 is fixedly assembled with an arc-shaped block 97. A telescoping spring 98 is fixedly assembled between the arc-shaped block 97 and the fixing block 63. The fixing block 63 on the connecting block 62 is moved toward the pressing slot 21 until the clamping block 96 is positioned on one side of the pressing slot 21. The arc-shaped block 97 bears a force, so that the guiding rod 95 moves on the fixing block 63. The telescoping spring 98 contracts, and the guiding rod 95 drives the clamping block 96 to clamp the surface of the flexible pipe 13 into the pressing slot 21, thereby increasing the stability between the flexible pipe 13 and the inlet connection 11 and the outlet connection 12 respectively.

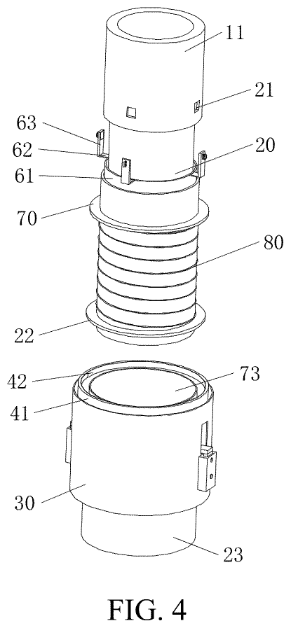

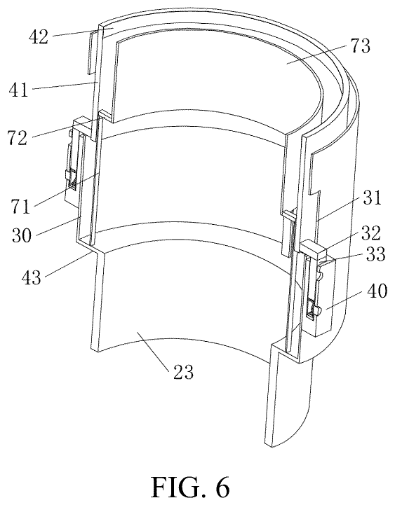

Referring to FIG. 4 and FIG. 6, a threaded sleeve 23 is screwed to the external thread 80. The threaded sleeve 23 is positioned between the first limiting ring 22 and the second limiting ring 70. A connecting disk 43 is fixedly assembled on one end of the threaded sleeve 23. A connecting pipe 30 is fixedly assembled on the connecting disk 43. The connecting pipe 30 is positioned on an outer side the second limiting ring 70. Multiple connecting rods 71 are fixedly assembled on the connecting disk 43. The connecting rods 71 are positioned on one side of the second limiting ring 70. One end of the connecting rod 71 is fixedly assembled with a mounting rod 72. The mounting rod 72 is positioned on one side of the second limiting ring 70. One end of each of the multiple mounting rods 72 is fixedly assembled with a pushing pipe 73. The threaded sleeve 23 is rotated and moved on the external thread 80, to make the connecting rod 71 drive the pushing pipe 73 on the mounting rod 72 to rotate and move, so that the pushing pipe 73 can push the connecting block 62 to move.

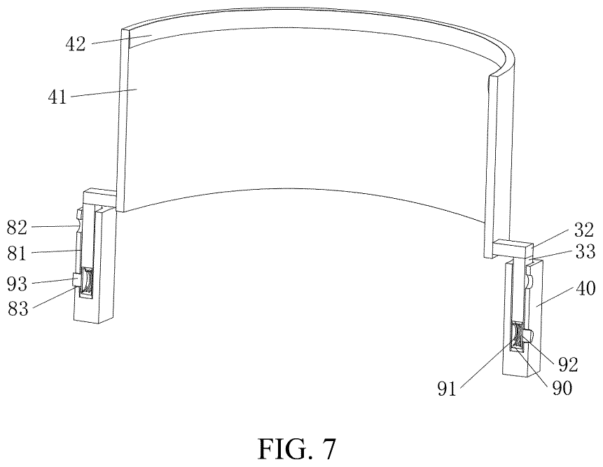

Referring to FIG. 6 and FIG. 7, moving ports 31 are symmetrically formed through the connecting pipe 30. Assembly blocks 40 are symmetrically and fixedly assembled on a surface of the connecting pipe 30. An inserting slot 81 is formed in one end of the assembly block 40. A first clamping hole 82 that communicates with the inserting slot 81 is formed in a surface of the assembly block 40. A second clamping hole 83 that communicates with the inserting slot 81 is formed in a surface of the assembly block 40. An inserting block 33 is fixedly assembled on the inserting slot 81. A guiding block 32 that moves at the moving port 31 is fixedly assembled at one end of the inserting block 33. An extension pipe 41 is fixedly assembled between two guiding blocks 32. The extension pipe 41 is positioned between a pushing pipe 73 and the connecting pipe 30. An inner convex ring 42 is fixedly assembled on an inner wall of the extension pipe 41. An operating cavity 90 is formed in the inserting block 33. An elastic spring 91 is fixedly assembled on the operating cavity 90. One end of the elastic spring 91 is fixedly assembled with a pushing plate 92. A telescoping button 93 is fixedly assembled on the pushing plate 92. The telescoping button 93 movably penetrates through the inserting block 33, and one end of the telescoping button 93 is separately clamped with only one of the first clamping hole 82 and the second clamping hole 83. The telescoping button 93 on the second clamping hole 83 is pressed, so that the elastic spring 91 contracts. The inserting block 33 is moved in the inserting slot 81, so that the telescoping button 93 is changed to be clamped in the first clamping hole 82. In this case, the inner convex ring 42 on the extension pipe 41 applies a force on the arc-shaped block 97, so that the arc-shaped block 97 drives the guiding rod 95 to move on the fixing block 63.

Working principle: When the inlet connection 11 and the outlet connection 12 are clamped into a flexible pipe 13 respectively, one end of the flexible pipe 13 is positioned in the clamping groove 20. The threaded sleeve 23 is rotated and moved on the external thread 80, to make the connecting rod 71 drive the pushing pipe 73 on the mounting rod 72 to rotate and move, so that the pushing pipe 73 can push the connecting block 62 to move. The connecting block 62 is pushed to move the movable pipe 61 on the inner cavity 50. One end of the inserting rod 52 moves inside of the mounting pipe 51, and the adjusting spring 60 is extended. In this case, the connecting block 62 and the inner wall of the clamping groove 20 clamp a surface of the flexible pipe 13, and the fixing block 63 on the connecting block 62 moves toward the pressing slot 21 until the clamping block 96 is positioned on one side of the pressing slot 21. The telescoping button 93 on the second clamping hole 83 is pressed, so that the elastic spring 91 contracts. The inserting block 33 is moved in the inserting slot 81, so that the telescoping button 93 is changed to be clamped in the first clamping hole 82. In this case, the inner convex ring 42 on the extension pipe 41 applies a force on the arc-shaped block 97, so that the arc-shaped block 97 drives the guiding rod 95 to move on the fixing block 63. The telescoping spring 98 contracts, and the guiding rod 95 drives the clamping block 96 to clamp the surface of the flexible pipe 13 into the pressing slot 21, thereby increasing the stability between the flexible pipe 13 and the inlet connection 11 and the outlet connection 12 respectively.

This specific embodiment is merely for explaining the present invention, but is not intended to limit the present invention. A person skilled in the art may make modifications to this embodiment as needed without creative contribution after reading this specification. However, as long as the modifications fall within the scope of the claims of the present invention, the modifications are protected by the patent law.

What is claimed is:

1. A pivotally-driven flexible membrane blood pump, comprising: a pump body (10), and an inlet connection (11) and an outlet connection (12) that communicate with the pump body (10), wherein ends of the inlet connection (11) and the outlet connection (12) communicate with flexible pipes (13) via fastening mechanisms; the fastening mechanism comprises a clamping groove (20) formed in the inlet connection (11); a plurality of pressing slots (21) that communicate with the clamping groove (20) are formed in a surface of the inlet connection (11); a first limiting ring (22) and a second limiting ring (70) are respectively fixedly connected to an outer side of the inlet connection (11); an external thread (80) is formed on the inlet connection (11) between the first limiting ring (22) and the second limiting ring (70); an inner cavity (50) is formed in the inlet connection (11); a plurality of mounting pipes (51) are arranged on an inner side of the inner cavity (50); an inserting rod (52) is movably connected to one end of the mounting pipe (51); an operating plate (53) is movably connected to one end of the inserting rod (52) far from the mounting pipe (51);
the inlet connection (11) is provided with a clamping mechanism;
the clamping mechanism comprises an adjusting spring (60) connected to the mounting pipe (51) and the inserting rod (52); two ends of the adjusting spring (60) are connected to the operating plate (53) and the inner cavity (50) respectively; a movable pipe (61) is mounted on the operating plate (53); one end of the movable pipe (61) movably penetrates the clamping groove (20); a plurality of connecting blocks (62) are fixedly connected to the movable pipe (61); a fixing block (63) is connected to an end of the connecting block (62) close to the inlet connection (11); the fixing block (63) is provided with a pressing mechanism;
the pressing mechanism comprises a placing groove (94) formed in the fixing block (63); a guiding rod (95) is slidingly connected in the placing groove (94); a clamping block (96) is connected to one end of the guiding rod (95); an arc-shaped block (97) is arranged at one end of the guiding rod (95) far from the clamping block (96); a plurality of telescoping springs (98) are fixedly connected between the arc-shaped block (97) and the fixing block (63);
the inlet connection (11) is further provided with an adjusting mechanism; the adjusting mechanism comprises a threaded sleeve (23) screwed to the external thread (80); the threaded sleeve (23) is positioned between the first limiting ring (22) and the second limiting ring (70); a connecting disk (43) is connected to one end of the threaded sleeve (23); a connecting pipe (30) is fixedly connected to the connecting disk (43); a plurality of connecting rods (71) are arranged on the connecting disk (43); a mounting rod (72) is connected to one end of the connecting rod (71) far from the connecting disk (43); and a pushing pipe (73) is connected to one end of the mounting rod (72) far from the connecting rod (71).

2. The pivotally-driven flexible membrane blood pump according to claim 1, wherein the connecting rod (71) is positioned on an outer side of the second limiting ring (70), and the mounting rod (72) is positioned on one side of the second limiting ring (70).

3. The pivotally-driven flexible membrane blood pump according to claim 1, wherein the inlet connection (11) is further provided with a pulling mechanism; the pulling mechanism comprises moving ports (31) symmetrically formed in the connecting pipe (30); assembly blocks (40) are symmetrically and fixedly connected to an outer side of the connecting pipe (30); an inserting slot (81) is formed in one end of the assembly block (40); and a first clamping hole (82) and a second clamping hole (83) that communicate with the inserting slot (81) are formed in a surface of the assembly block (40).

4. The pivotally-driven flexible membrane blood pump according to claim 3, wherein the pulling mechanism comprises an inserting block (33) connected to the inserting slot (81); a guiding block (32) is arranged at one end of the inserting block (33); the guiding block (32) performs movable assembly corresponding to the moving port (31); an extension pipe (41) is jointly connected between the two guiding blocks (32); and the extension pipe (41) is positioned between the pushing pipe (73) and the connecting pipe (30).

5. The pivotally-driven flexible membrane blood pump according to claim 4, wherein the pulling mechanism further comprises an inner convex ring (42) fixedly connected to an inner wall of the extension pipe (41); an operating cavity (90) is formed in the inserting block (33); an elastic spring (91) is fixedly connected to the operating cavity (90); a pushing plate (92) is arranged at one end of the elastic spring (91); a telescoping button (93) is mounted on the pushing plate (92); and the telescoping button (93) penetrates and is connected into the inserting block (33).
